(19) United States
(12) Patent Application Publication
(10) Pub. No.: US 2016/0198991 A1
(43) Pub. Date: Jul. 14, 2016

(54) Universal Sampling Cap

(71) Applicant: NOBLE HOUSE GROUP PTY. LTD., Chelsea Heights (AU)

(72) Inventor: Chris WHELAN, Leichhardt (AU)

(21) Appl. No.: 14/912,095

(22) PCT Filed: Aug. 13, 2014
(86) PCT No.: PCT/AU2014/000805

§ 371 Date: Feb. 15, 2016

(30) Foreign Application Priority Data
Aug. 13, 2013 (AU) 2013903039

(51) Int. Cl. A61B 5/15; A61B 10/00; A61B 5/154

(52) U.S. Cl. CPC A61B 5/150351; A61B 5/154; A61B 10/0096

(57) ABSTRACT

A sampling port (10) has a generally tubular body (12) defined by a wall (18) arranged about an axis (42) which defines a space (21) for a sampling vial (72, 102). The wall (18) has a waisted region (60) intermediate a base (16) and an open end (20). The wall (18) is expandable radially in at least the waisted region (60) to accommodate different sized sampling vails (72, 102).

### FIELD OF INVENTION

This invention relates to medical sampling ports and more particularly providing a sampling port that can accommodate different diameter sampling bottles and/or vials. For ease of reading the term “sampling vial” alone is used in this specification and the term “sampling vial” is to be interpreted to include both sampling vial and sampling bottle.

### BACKGROUND

Medical sampling ports are devices that allow a sample of fluid to be withdrawn, typically into a sampling vial. Typical sampling ports are tubular with one end closed to form a base and the other end open. A cannula is centrally mounted on the base and extends within the port toward the open end. The end of the cannula ends short of the end of the sampling port and so is not exposed. A sampling vial may be passed into the open end and impaled on the cannula.

It is desirable that the sampling vial when inserted into the sampling port is prevented from significant sideways movement, as this may result in damage to the cannula or incomplete penetration of the cannula into the sampling vial.

Sampling vials generally come with portions to be inserted in to a sampling port in two or more different maximum diameters. It is desirable that these different diameters are readily accommodated by the sampling port without the need for separate guides or the like that are inserted or removed from the sampling port to accommodate the different diameters.

Further, it is desirable to have the free end of the cannula recessed a significant distance from the open end of the sampling port so as to reduce or avoid accidental needle stick injury.

### SUMMARY OF THE INVENTION

In one broad form the invention provides a sampling port having:
a body having a space having a longitudinal direction, open at one end and adapted to receive a sampling vial;
a cannula having an axis and extending longitudinally along the space with a pointed end facing the open end, whereby a sampling vial passed into the space from the open end may be impaled on the cannula;
the space defined by a wall arranged about the axis and extending generally axially;
the wall having a waisted region intermediate the base and free end so as to define a waisted space region, having, in axial end view, a cross section smaller than cross sections at the open end and a base end;
the wall being radially expandable in at least the waisted region whereby the size of at least the waisted space region is expandable.

The wall may be expandable by being formed of an elastic material or by being formed by one or more wall members that allow expansion by elastic deflection, whether by elastic deflection within their own structure or elastic deflection of the member relative to the rest of the body, or a combination of both.

In another broad form the invention provides a sampling port having:
a body having a space having a longitudinal direction, open at one end and adapted to receive a sampling vial;
a cannula having an axis and extending longitudinally along the space with a pointed end facing the open end, whereby a sampling vial passed into the space from the open end may be impaled on the cannula;
at least part of the space defined by at least one movable wall portion, the at least one movable wall portion movable at least radially toward or away from the axis;
the at least one movable wall portion guiding a vial of a first size when inserted into the open end;
the at least one movable wall portion movable to allow a vial of a size larger than the first size to be inserted into the space and guided by the at least one movable wall portion onto the cannula.

Movement of the wall portion is preferably caused by the vial as it is inserted into the sampling port. However, the invention includes movement by other ways.

In another broad form the invention provides a sampling port having:
a body having a space having a longitudinal direction, open at one end and adapted to receive a sampling vial;
a cannula having an axis and extending longitudinally along the space with a pointed end facing the open end, whereby a sampling vial passed into the space from the open end may be impaled on the cannula;
the space defined by a plurality of wall members arranged about the axis and extending generally axially;
at least part of each wall member being movable radially away from the axis to increase the size of the space defined by the wall members.

The body may have a base portion and the or each wall member may extend from the base portion.

The or each wall member may flex, hinge, bend or otherwise deflect relative to the base portion.

The or each wall member may have base end portion, a free end portion at or adjacent the open end of the space and an intermediate portion located between the base and free end portions.

Part of the intermediate portion may be spaced a first distance from the axis.

Part of the free end portion may be spaced a second distance from the axis.

Part of the base end portion may be spaced a third distance from the axis.

Preferably the first distance is less than the second distance. Preferably first distance is less than the third distance. The second and third distances may be the same.

In a preferred implementation the or each wall member is curved along its length so that the intermediate portion is nearer the axis than the base and free end portions.

Preferably an axially extending slot is provided in at least one wall member.

Where there is a plurality of wall members, preferably each wall member has an axially extending slot. An axially extending slot may be provided in some but not all of a plurality of wall members.

The axially extending slot preferably passes through the waist region.

The wall members are preferably flexible such that the waist region may deflect from being concave to being convex.

A ring member may extend around the axis and the free end portions of any wall member(s) to limit radial movement of the free end(s).

In a preferred implementation the ring member is connected to at least one free end at a connection location. Where there is a plurality of wall members the ring member may be connected to all of the free ends at a respective connection location or only some of the free ends. If not connected to a free end, preferably the ring member bears against the free end.

The ring member may be elastic. Preferably the ring member is flexible and has a length greater than the circumference of a circle centered on the axis and passing through the connection locations.

Preferably the ring member comprises a plurality of peaks and troughs around the free end of the body.

Preferably the ring member comprises a plurality of curved portions. Preferably adjacent curved portions are curved in opposite direction.

Preferably adjacent curved portions are joined to each other at a point of inflection.

In cross section, each curved portion may be part of an ellipse, oval or circle. However, at least the curved portion may be a freehand spline rather than a mathematically defined line.

Preferably the ring member is tangent to the free end portions at a respective connection location.

Preferably the ring member connects to a free end portions at a trough in the ring member. Where there are multiple connection locations, preferably there are two peaks and one trough between connection locations.

These and other aspects of the invention will be apparent from the following non-limiting description of a preferred implementation of the invention with reference to the attached representations.

### BRIEF DESCRIPTION OF THE DRAWINGS

FIG. 1 is a perspective view from below of a sampling port according to a first implementation of the invention.

FIG. 2 is a perspective view from above of the sampling port of FIG. 1.

FIG. 3 is a side view of the device of FIG. 1.

FIG. 4 is an end view from below of the device of FIG. 1.

FIG. 5 is a side view of the sampling port of FIG. 1 in use with a first size vial.

FIG. 6 is a cross sectional side view taken along line AA in FIG. 5.

FIG. 7 is a side view of the sampling port of FIG. 1 in use with a second size vial with the vial partially inserted.

FIG. 8 is a cross sectional side view taken along line BB in FIG. 7.

FIG. 9 is a perspective view from above of the arrangement of FIG. 7.

FIG. 10 is a side view of the sampling port of FIG. 1 in use with a second size vial with the vial fully inserted.

FIG. 11 is a cross sectional side view taken along line CC in FIG. 10.

FIG. 12 is a perspective view from above of the arrangement of FIG. 10.

### DETAILED DESCRIPTION OF PREFERRED AND OTHER EMBODIMENTS

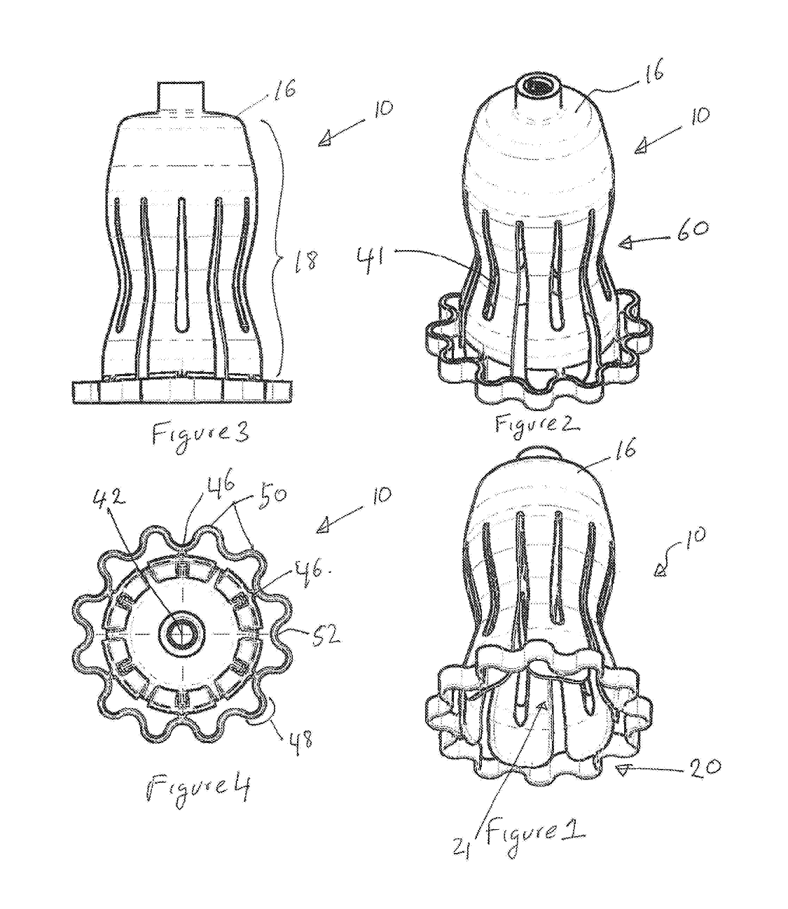

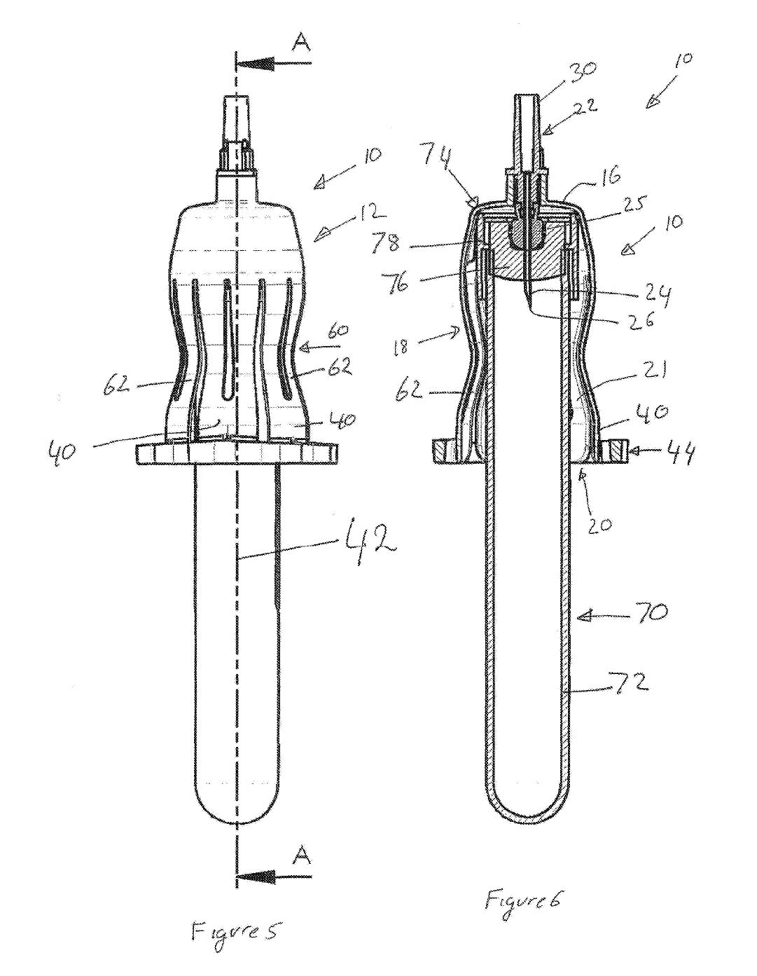

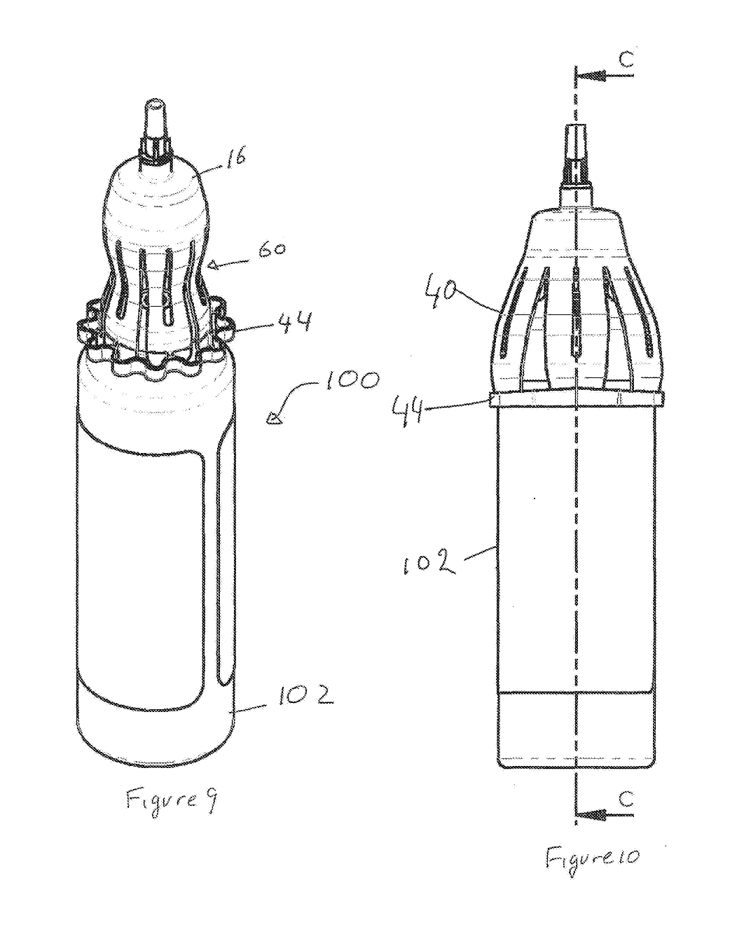

Referring to FIGS. 1 to 12 there is shown a sampling port 10 according to one implementation of the invention.

The sampling port 10 has a generally tubular body 12. The body 12 has a base 16 at one end and a side wall 18 that extends away from the base 16. The side wall 18 defines an open end 20 and a space 21. Mounted on the base 16 is a needle assembly 22 having a cannula 24. The cannula 24 is mounted generally centrally and extends along the body toward the open end 20. The free end 26 of the cannula 24 is located away from the open end 20. A rubber sheath 25 surrounds the cannula 24.

The interior of the cannula 24 communicates with a connector or piercing device of needle assembly 22 so that fluid may pass from the connector to the cannula 24 or vice versa. In the implementation shown the connector is a male luer connector 30 and the assembly 22 is a screw fit into base 16. If desired the base 16 and assembly 22 may be formed together with the cannula mounted in the integral base. The type of connector or piercing device is not critical and variations may be used, including a male or female luer, locking luer, multi-sample (double-ended) needle, blunt cannula, spike etc.

The wall 18 of the body 12 has six elongate flexible wall members 40 spaced about the axis 42 of the body. Each wall member 40 extends from the base portion 16 to the open end 20. In this implementation each wall member is attached to a ring member 44 at the open end. The ring member extends around the open end and preferably is attached to each wall member at connection points 46. If not connected to a free end, preferably the ring member bears against the free end.

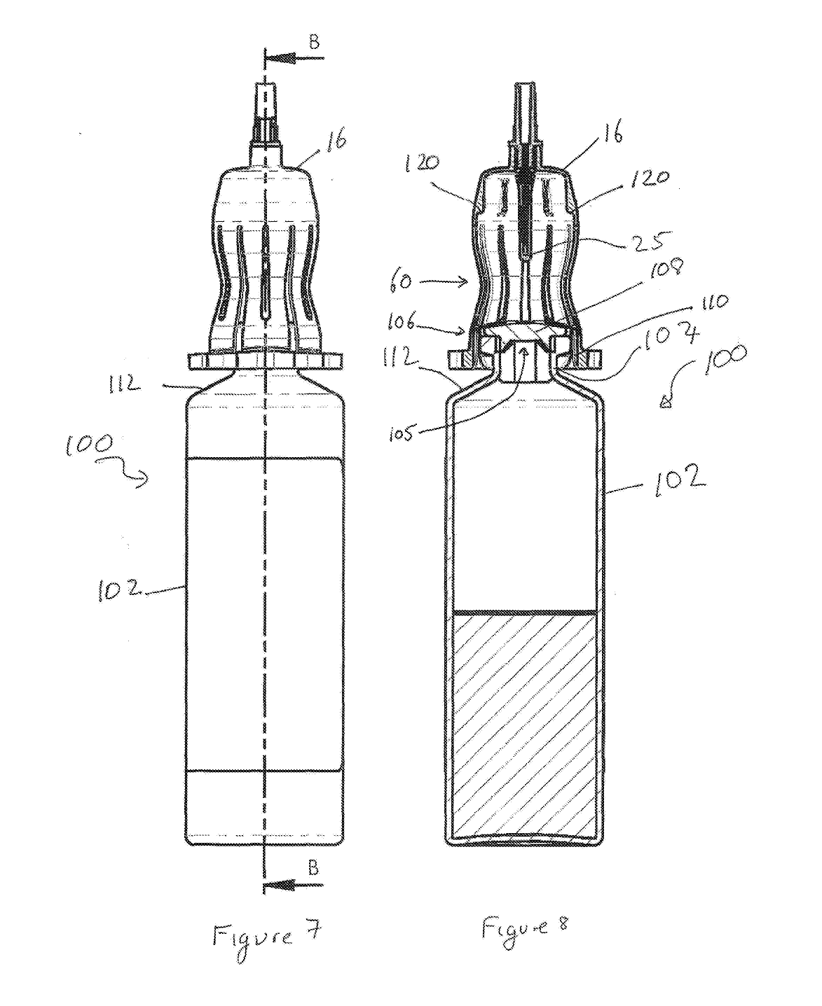

As seen in FIG. 4, each wall member 40 is curved in end view so the free ends extend generally circumferentially about the axis.

The expanded length of the ring member 44 between adjacent connection points 46 is greater than the circumferential distance between them. In this implementation the ring member 44 is formed with a series of curved portions 48 that subtend an angle of 180 degrees in alternating directions. Use of segments that subtend an angle of 180 degrees allows the relevant segments 46 to be tangent to the wall members 40.

In this implementation there are two peaks 50 and one trough 52 between each connection point 46. If desired there could be a single peak and no trough between connection points. As the number of peaks between adjacent connection points increases the radial extent reduces for the same length between adjacent connection points.

The ring, wall members and base are preferably all formed as a single moulding but may be formed of separate components.

The wall members 40 are flexible and may be flexed, hinged, bent or otherwise deflected radially outwards so that the free ends of the wall members are further away from the axis and the effective circumference of the open end is increased.

The tubal body 12 is waisted, in that it has a reduced diameter portion 60 between the base portion 16 and the open end 20. The internal diameter of the open end 20 may be the same diameter as that of the base portion 16.

The body 12 thus has an open end with diameter D1 and a minimum diameter at waist 60 of D2.

This waisting is achieved by having each wall member 40 curve inwards toward the axis.

In the implementation shown the waist region 60 of the body 12 is between the open end 20 and the free end 25 of the cannula. The waist region 60 thus serves to centralise a stationary vial having a diameter approximately D2 when partially inserted but not yet impaled on the cannula.

In use, a vial having a maximum diameter less than D1 may be inserted into the body 12. Ideally the device is used with vials having a maximum diameter greater than D2 but this is not essential.

Referring to FIGS. 5 and 6, a vial 70 comprising tube 72 and closure 74 has been inserted and impaled on the cannula 24. In this case the closure comprises a pierceable bung 76 retained by cap 78 that extends over part of the tube 72. Both the tube 72 and cap 78 have a diameter less than D1. Ideally their diameters are greater than D2. In use the vial is inserted until cap 78 contacts sloping waisted portions 62 of wall members 40. The vial 70 will be centered by the wall members 40 as the vial is inserted. As the vial is inserted the cap, being of diameter greater than D2 of waist 60, causes the waisted portion of wall members to flex outwards elastically, expanding the diameter of waist 60 and allowing the cap and tube to pass. In the example shown, the diameter of the tube 70 is less than that of cap 78 and after the cap has passed waist 60, the wall members move radially inwards and bear against the tube 72, thus holding and maintaining the vial 70 centered. Withdrawal of the vial causes the wall members 40 to flex elastically outwards to allow the cap 78 to pass the waist.

The sampling port may also be used with containers with parts having a diameter greater than D1 of the opening and in particular sampling bottles, as shown in FIGS. 7 to 11. In these examples the sampling bottle 100 has a bottle body 102 with a diameter greater than D1 with a neck 104 opening at mouth 105 closed by closure 106. The closure comprises a pierceable bung 108 and cap 110.

The diameter of cap 110 is less than D1 but greater than D2.

As best seen in FIG. 8, as the sampling bottle is inserted into the port the cap engages the waisted portion of the body 12 and is centered over the cannula 24. Insertion continues until the free ends 45 of wall members 40 engage sloping end portion 112 of bottle body 102. Continued insertion causes the wall members to flex about base portion 16 so as to increase the diameter of opening 20, with the segments 50, 52 of the ring member 44 becoming less curved and the ring member 44 as a whole becoming less “rippled” as the distance between adjacent connection points 46 increases.

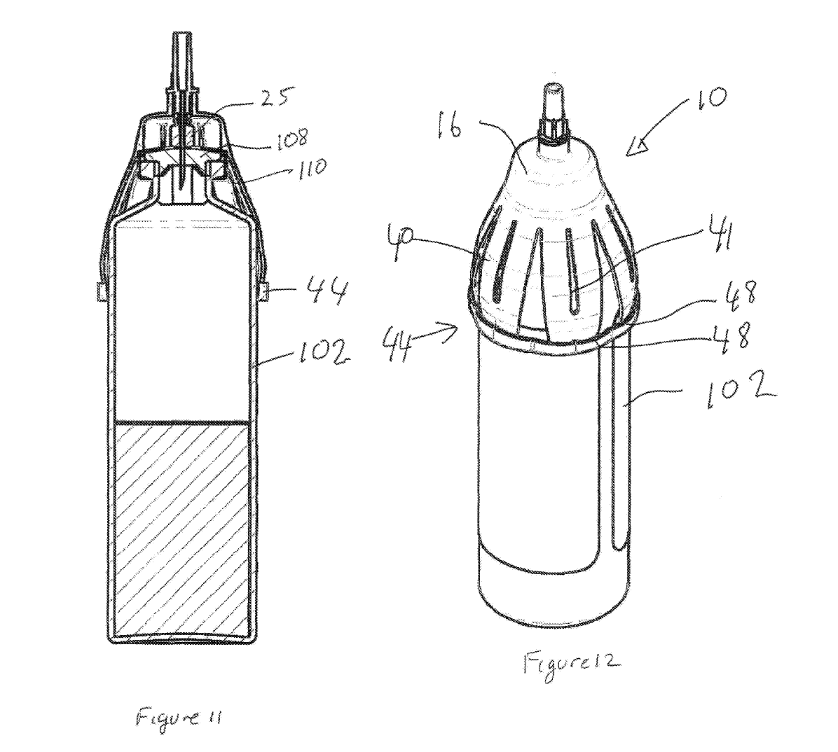

The sampling bottle may thus be fully inserted and impaled on the cannula 24, as shown in FIGS. 10 to 12, with the wall members extending over the bottle 102. The base portion 16 has stop members 120 that limit insertion of the sampling bottle 100 into the port.

The ring member 44 serves a number of functions. The main function is to limit the flexing of the wall members 40 relative to base 16. As seen in FIG. 12, maximum flexing of the wall members 40 is limited to when the ring member is almost circular. In the absence of the ring member 44 the wall members may tend to flex about the base portion by rotation about a hinge line, so presenting a flower petal arrangement. Such hinging may result in non-elastic flexing, so that the wall members will not return to the initial configuration when the bottle is removed and leaving the cannula with no protection. A secondary aspect is that the ring member provides additional “closing” force to return the wall members to the original position when the bottle is removed.

It will be appreciated that the ring member 44 does not need to be connected to every wall member 40, although this is preferred. The ring like nature of the ring member ensures that the outward movement of the wall members is controlled even if not connected. Radially outward movement of the wall members will result in the free end contacting and/or urging the ring member radially outward even if there is no actual connection between the ring member and the respective wall member.

The wall members are flexible along their length and thus do not just hinge, flex or bend about the base portion. In the preferred implementation the waisted portion of the wall members may be deflected, bent or straightened relative to the end portions to accommodate the bottle body. As seen most clearly in FIG. 11, the wall members flex so that the inner surface of each is concave around the bottle body 102, compared to being convex in the waisted position.

In the implementation shown each wall member 40 is provided with an elongate slot 41. This slot assists in the wall members flexing to have a larger effective diameter but may be omitted. In the implementation shown there are six wall members 40, each with a slot. If desired the sampling cap may be provided with more or less wall members, with or without slots. For example a sampling cap may be provided with twelve wall members, each without a slot but roughly half the width of the wall members 40 shown in the representations.

The implementation shown provides a waisted tubular body that expands by having movable wall members that are relatively inelastic. Whilst accommodation of “small” vials is achieved by elastic flexing of the waisted portion of the wall members, accommodation of “large” vials is achieved by gross movement of the wall members. It will be appreciated that forming the body of a more elastic material may allow the wall of the tubular body to expand elastically to accommodate “large” vials. Accordingly the tubular body could, for example, be formed of a rubber or synthetic rubber having appropriate elasticity and stiffness, without the need for separate wall members extending around the axis.

Unless the context clearly requires otherwise, throughout the description and any claims the words “comprise”, “comprising”, and the like are to be construed in an inclusive sense as opposed to an exclusive or exhaustive sense; that is to say, in the sense of “including, but not limited to”.

The features of the invention described or mentioned in this document may be combined in any combination of features where features are not mutually exclusive.

It will be apparent to those skilled in the art that many obvious modifications and variations may be made to the embodiments described herein without departing from the spirit or scope of the invention.

1. A sampling port having:
a body having a space having a longitudinal direction, an open end and adapted to receive a sampling vial;
a cannula having an axis and extending longitudinally along the space with a pointed end facing the open end, whereby a sampling vial passed into the space from the open end may be impaled on the cannula;
the space defined by a wall arranged about the axis and extending generally axially;
the wall having a waisted region intermediate a base end and the open end so as to define a waisted space region, having, in axial end view, a cross section smaller than cross sections at the open end and the base end;
the wall being radially expandable in at least the waisted region whereby the size of at least the waisted space region is expandable.

2. The sampling port of claim 1 wherein the wall is formed of an elastic material or formed by at least one wall member that allows expansion by elastic deflection, including by elastic deflection within the structure of the at least one wall member or elastic deflection of the at least one wall member relative to the rest of the body, or a combination of both.

3. (canceled)

4. (canceled)

5. (canceled)

6. (canceled)

7. (canceled)

8. The sampling port of claim 1 wherein the or each wall member has a base end portion, a free end portion at or adjacent the open end of the space and an intermediate portion located between the base and free end portions.

9. (canceled)

10. (canceled)

11. (canceled)

12. (canceled)

13. The sampling port of claim 8 wherein the or each wall member is curved along its length so that the intermediate portion is nearer the axis than the base end portion and the free end portion.

14. The sampling port of claim 8 wherein the intermediate portion is concave and may deflect from being concave to being convex.

15. The sampling port of claim 14 wherein the or each wall member is movable so that at least a part of the intermediate portion is further away from the axis than a part of the base end portion.

16. (canceled)

17. (canceled)

18. (canceled)

19. (canceled)

20. The sampling port of claim 1 comprising at least two wall members and at least one connecting member connected to the at least two wall members to limit radial movement of the free end of the wall.

21. The sampling port of claim 1 including a ring member extending around the axis and the wall adjacent the open end at at least one limit location to limit radial movement of the free end of the wall.

22. The sampling port of claim 21 wherein there are multiple limit locations.

23. The sampling port of claim 21 wherein the ring member is connected to the wall at at least one limit location.

24. (canceled)

25. (canceled)

26. (canceled)

27. The sampling port of claim 23 wherein the ring member is flexible and has a length greater than the circumference of a circle centered on the axis and passing through at least one limit location.

28. The sampling port of claim 27 wherein the ring member comprises a plurality of peaks and troughs around the free end of the body.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. The sampling port of claim 1 wherein the waisted region is intermediate the pointed end of the cannula and the open end.

44. A sampling port having:
a body having a space having a longitudinal direction, an open end and adapted to receive a sampling vial;
a cannula having an axis and extending longitudinally along the space with a pointed end facing the open end, whereby a sampling vial passed into the space from the open end may be impaled on the cannula;
the space defined by a wall arranged about the axis and extending generally axially;
the wall having a waisted region intermediate a base end and the open end so as to define a waisted space region, having, in axial end view, a cross section smaller than cross sections at the open end and the base end;
the wall being radially expandable in at least the waisted region whereby the size of at least the waisted space region is expandable,
wherein
the wall comprises a plurality of wall members;
each wall member has a base end portion, a free end portion at or adjacent the open end of the space and an intermediate portion located between the base and free end portions;
each intermediate portion is concave and may deflect from being concave to being convex, and
at least one connecting member connected to at least two wall members to limit radial movement of the free end of the wall.

45. The sampling port of claim 44 wherein the at least one connecting member comprises a ring member extending around the axis and the wall adjacent the open end at at least one limit location.

46. The sampling port of claim 45 wherein the ring member comprises a plurality of peaks and troughs around the free end of the body.

47. The sampling port of claim 44 wherein the waisted region is intermediate the pointed end of the cannula and the open end.
